(19) United States
(12) Patent Application Publication
(10) Pub. No.: US 2019/0029839 A1
(43) Pub. Date: Jan. 31, 2019

(54) KIT FOR FOOT ARTHROPLASTY

(71) Applicant: BIOTECH ORTHO, Salon de Provence (FR)

(72) Inventor: Jean-Alain COLOMBIER, BALMA (FR)

(21) Appl. No.: 16/152,517

(22) Filed: Oct. 5, 2018

Related U.S. Application Data
(63) Continuation of application No. 15/324,782, filed on Jan. 9, 2017, now abandoned.

(30) Foreign Application Priority Data
Jul. 10, 2014 (FR) 1456673

(51) Int. Cl. A61F 2/42; A61B 17/16; A61F 2/30

(52) U.S. Cl. CPC A61F 2/4225; A61B 17/1682; A61F 2002/30616; A61F 2002/4228; A61F 2002/4233; A61B 17/1615

(57) ABSTRACT

Disclosed is an assembly for foot arthroplasty, comprising a plurality of successively larger implants (10, 20, 30, 40) and a single drill (60); each implant includes, relative to a central portion (11), a proximal pin (12, 22, 32, 42) and a distal pin (13, 23, 33, 43) that is shorter than the proximal pin, said pins and said drill being tapered by 2 to 6°.

The invention concerns a set of implants for performing foot arthroplasty, that is to say to surgically reform a joint of the foot, whether it be interphalangeal or metatarsophalangeal.

Various techniques are known for reforming a joint. Thus document WO-2010/079288 discloses an orthopaedic implant used in arthroplasty of the finger comprising a first member provided to be implanted in a proximal phalanx and a second member provided to be implanted in an adjacent distal phalanx, each member comprising a stem for implantation in the bone and an interphalangeal joint head, the head of the first member having a biconvex joint surface with a central valley and the head of the second member having a biconcave joint surface arranged to cooperate with said biconvex surface and comprising a central crest.

In the case of foot arthroplasty, it is known to use a member formed from two stems extending in opposite directions from each other from a median ball-shaped part. The stems are of different lengths, the stem that is provided to be implanted in a phalanx referred to as distal—referred to as distal stem—being shorter than the stem provided to be implanted in a phalanx referred to as proximal—referred to as proximal phalanx (it is to be recalled that the distal phalanx is further from the ankle than the proximal phalanx and is thus shorter than it). The material constituting such a one-piece implant is chosen so as to have compatible flexibility with that sought for the joint to reconstitute (silicone in practice).

When such an implant is implanted, the surgeon begins by incising the tendon actuating the movement of the distal phalanx relative to the proximal phalanx and a bit is used to bore a canal in one then the other of the phalanxes before inserting the corresponding stem therein.

In practice a choice must be made between several sizes of implants according to the configuration of the joint to be reformed; it can indeed be understood that the larger the joint to reconstitute, the larger must be the size of the implant; there are currently between 2 and 5 different sizes within a set of implants. By way of example, if two implants of successive size are considered, it is possible to have stems of the same diameters and the same lengths, but median parts of different diameters, or on the contrary median parts of the same diameters separating stems of different diameters and lengths. According to the diameter of the implant chosen, the surgeon chooses a bit of appropriate diameter and bores a canal of length hardly greater than that of the corresponding stem.

Thus, a set of implants conventionally comprises a plurality of implants as well as a plurality of bits; furthermore, as the implants are usually obtained by molding, as many different molds are required as there are implants in the set considered.

The invention is directed to simplifying the constitution of a set of such implants for foot arthroplasty, or even its its manufacture.

To that end it provides a set of implants for foot arthroplasty comprising a plurality of implants of increasing sizes and a single bit, each implant comprising, starting from a median portion, a proximal stem and a distal stem shorter than the proximal stem, these stems and this bit having equal conical tapers comprised between 2 and 6°; preferably, the stems and the bit have a conical taper of 4.8° approximately.

Preferably, the distal stem of an implant has the same length as the proximal stem of another implant, which simplifies the formation of the molds for manufacture of the implants of a same set.

Advantageously, the bit comprises on its working stem at least one visual marker defining, from the point of that bit, a penetration distance equal to the length of at least one distal or proximal stem of an implant of said set. This enables the surgeon to make a good estimate of the depth to which he must bore a canal which is to receive a given stem of a chosen implant.

Preferably, the implants, classified in increasing order of size, each have (except for the largest) a proximal stem of which the length is equal to that of the distal stem of the next implant.

Advantageously, the bit comprises a visual marker on its working stem for each of the different lengths of the stems of the implants of said set. However, a number of markers equal to N+1 suffices if N is the number of implants.

Advantageously, with each of the different lengths of stem there is associated a specific texture present on the stems having that length and between the visual marker associated with that length and the preceding marker. This facilitates the perception by the surgeon of the marker to use when boring a canal which is to receive a given stem of a chosen implant.

Preferably, the median parts are ball-shaped, spherical, oval or ellipsoidal, with diameters (or transverse dimensions) which advantageously increase on going from a given implant to an implant of larger size.

Objects, features and advantages of the invention appear from the following description, given by way of illustrative non-limiting example, with reference to the accompanying drawing in which:

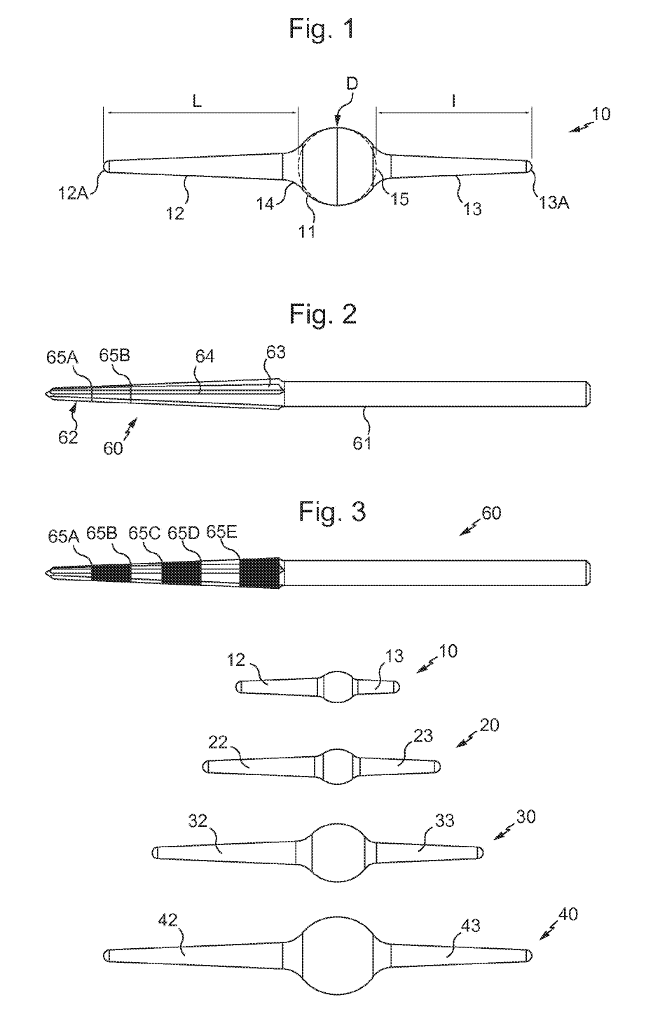

FIG. 1 is a side view of an implant forming part of a set in accordance with the invention,

FIG. 2 is a side view of an implant forming part of a set in accordance with the invention, and

FIG. 3 is a side view of a set of four implants and of the associated bit.

FIG. 1 represents an implant 10 comprising a median part 11 formed from a ball of a given diameter D and two stems 12 and 13 extending away from each other from that median part. These two stems are of different lengths, the stem 12, referred to as proximal stem (provided to be implanted in a proximal phalanx) being longer than implant 13, referred to as distal stem (provided to be implanted in a distal phalanx). The ratio between the length “I” of the distal stem and the length of the proximal stem “L” is for example comprised between ½ and ⅘. These lengths are measured between the end 12A or 13A (in practice rounded) of the stems and the median part 11 here likened to a real sphere (the fictional contour of this sphere is represented in dashed line in FIG. 1).

The stems are joined to the median part by transition zones 14 and 15;

According to the invention, each of the stems has a conical shape with the same conical taper; this conical taper is in practice chosen between 2 and 6°, for example 4.8°, a value which enables the drilling to be facilitated by the conical taper, without however involving a high increase in the entry diameter of the canal so formed.

Such an implant is in practice formed from a biocompatible material, for example medical silicone.

The bit, designated by the reference 60, comprises, beyond a holding portion 61, a working stem 62 comprising longitudinal grooves 63 (with possibly a helical component) bordering cutting ridges 64; this working stem has the same conical taper as the stems of the implant 10. On this working stem markers 65A and 65B are advantageously visible, which are situated relative to the point of the working stem at distances to which the bit must penetrate into the bone to enable proper location of a stem in the bone of the phalanx which is to receive it (marker 65A here corresponds to a depth of penetration useful for the implantation of the distal stem and marker 65B corresponds to the depth of penetration useful for the implantation of the proximal stem. Of course, it is possible for these markers not to be present, the surgeon being able to define by himself the depth of boring from the dimensions of the implant to be fitted.

FIG. 3 represents an example of a set of implants comprising implants in accordance with that of FIG. 1 and a single bit in accordance with FIG. 2. The various implants have stems having a same conical taper, equal to that of the bit. The number of implants within a set is here arbitrarily chosen to be four.

According to a preferred particular feature of the implants of the set, the proximal stem of the smallest implant has the same length as the distal stem of the implant of immediately greater size. In other words, if the four implants represented in FIG. 3 are denoted 10, 20, 30 and 40, the proximal stem 12 of the implant 10 has the same length as the distal stem 23 of the implant 20, the proximal stem 22 of that implant 20 has the same length as the distal stem 33 of the implant 30, and the proximal stem 32 of that implant 30 has the same length as the distal stem 43 of the implant 40.

In FIG. 1, which is directed to showing the general geometry of the implants of a set in accordance with the invention, an arbitrary choice has been made whereby that the implant 10 of FIG. 3 is indicated rather than, for example, the implant 40.

It can be understood that an advantage of such a graduation of the implant stems is that, in particular, the molds for molding a given implant adopts, for at least one of its stems, the geometry of a part of the mold associated with an implant of immediately greater or smaller size; this results in a simplification in the manufacture of the molds.

Another advantage of this graduation is that the bit can comprise a limited number of visual markers to assist the surgeon in identifying the level of penetration to which the bit is to be advanced; the first marker 65A corresponds to the depth of penetration of the bit to bore the canal which is to receive the distal stem of the smallest implant, i.e. the implant 10; however the second marker 65B corresponds to the depth of penetration of the bit to bore the canal which is to receive the proximal stem of that implant 10, but also the canal which is to receive the distal stem of the implant of immediately greater size, i.e. the implant 20; similarly, the marker 65C corresponds to the depth of penetration associated with the proximal stem of the implant 20 as well as of the distal stem of the implant 30, and the marker 65D corresponds to the depth of penetration for the proximal stem of the implant 30 and of the distal stem of the implant 40. Furthermore, the marker 65E corresponds to the depth of penetration of the canal which is to receive the proximal part of the largest implant.

It can thus be understood that there is a number of markers only just equal to N+1 if N is the number of implants.

To further facilitate the ergonomics of the kit, each stem can advantageously have a specific texture (this may be a color), reproduced on the bit. In such a case, advantageously, the texture of the distal stem 13 of the smallest implant 10 may be reproduced on the bit between its end and the first marker 65A, the texture in common to the proximal stem 12 of the first implant and to the distal stem 23 of the implant 20 may be reproduced between the first and second markers 65A and 65B, the texture in common to the proximal stem 22 of the second implant 20 and to the distal stem 33 of the third implant 30 may be reproduced between the markers 65B and 65B, the texture in common to the proximal stem 32 of the third implant and to the distal stem 43 of the fourth implant 40 may be reproduced between the markers 65C and 65D and the texture of the proximal stem 42 of this last implant may be reproduced between the markers 65D and 65E. If the surgeon wishes to implant one of the stems of one of the implants in a phalanx, he can thus know that he must extend the penetration of the bit until the portion of the bit having the texture of the stem in question disappears.

Such a succession of textures is, arbitrarily, shown in FIG. 3 (on the bit alone and not on the implants) by a succession of light and black zones; as a variant these textures can all be different. It can however be understood that, in a simplified version, it may be chosen not to seek to master the surface state and/or color of the stems of the various implants.

Advantageously, the lengths of the stems have approximately equal differences; thus by way of example, the lengths have a difference of the order of 4 to 6 mm, for example 5 mm (with for example a succession of lengths of the type 6, 11, 15, 20, 25 mm).

As regards the median parts, they preferably present a progression, chosen to be compatible with the variations in diameter of the stems with regard to these median parts (by way of example, the diameters will staggered as follows: 5-5.5-8.5-11 mm).

In practice, prior to actually implanting an implant, a surgeon makes tests using implants which are dummies (in the sense that they are representative of the real implants without being them), so as to ensure that the tests do not risk degrading the implant which will finally be implanted; these trial implants, sometimes called ghost implants, have the same progression of length (as regards the stems) or diameter (as regards the median parts).

In the simplified version of a set in accordance with the invention, it is possible for there to be identity between the length of a proximal stem of one implant and that of the distal stem of another implant for only some of the implants, in particular when the depth of penetration of the bit merits being viewed only for some of the implants.

Furthermore, it is possible for this identity of length not to correspond to implants following in the succession of the implants by size; thus, it is possible to provide for the proximal stem of the smallest implant to have the same length as the distal stem of not the second implant, but of the third, for the proximal stem of the second implant to have the same length as the distal stem of the fourth implant, and so forth, which can have the advantage of obtaining greater flexibility in the choice of the implant to implant.

It is to be noted that the invention, with one of the stems and bit of conical form (with a cross-section which is circular with a diameter that varies continuously, and linearly in practice) to be advantageously combined, if desired, with a one-piece character of each stem; such a one-piece character is not incompatible with the fact that these stems may, where appropriate, present a certain degree of flexibility.

1-9. (canceled)

10. A kit for foot arthroplasty comprising a plurality of implants having increasing sizes and a single bit, each implant of the plurality of implants comprising, starting from a median portion, a proximal stem and a distal stem shorter than the proximal stem,
wherein the proximal stem of each implant of the plurality of implants, the distal stem of each implant of the plurality of implants, and the bit have respective conical tapers which are all equal to a same value that is between 2 and 6°.

11. A kit according to claim 10, wherein said same value is 4.8°.

12. A kit according to claim 11, wherein the bit comprises a working stem provided with at least one visual marker defining, from a point of that bit, a penetration distance equal to the length of at least one distal or proximal stem of an implant of the plurality of implants.

13. A kit according to claim 11, wherein each of the plurality of implants, except for the largest, when classified in increasing order of size, have the proximal stem which has a length equal to that of the distal stem of the next implant.

14. A kit according to claim 11, wherein the median portion of each of the plurality of implants is ball-shaped.

15. A kit according to claim 10, wherein the bit comprises a working stem provided with at least one visual marker defining, from a point of the bit, a penetration distance equal to a length of at least one distal or proximal stem of an implant of the plurality of implants.

16. A kit according to claim 15, wherein the median portion of each of the plurality of implants is ball-shaped.

17. A kit according to claim 10, wherein each of the plurality of implants, except for the largest, when classified in increasing order of size, have the proximal stem which has a length equal to that of the distal stem of the next implant.

18. A kit according to claim 17, wherein the median portion of each of the plurality of implants is ball-shaped.

19. A kit according to claim 17, wherein the bit comprises a working stem provided with a visual marker for each of the different lengths of the proximal and distal stems of the implants of the plurality of implants.

20. A kit according to claim 19, wherein the median portion of each of the plurality of implants is ball-shaped.

21. A kit according to claim 19, wherein each of the different lengths of the proximal and distal stems of the implants of the plurality of implants is associated with a specific texture which is present on the proximal and distal stems having that length and which is present between the visual marker associated with that length and the preceding marker.

22. A kit according to claim 21, wherein the median portion of each of the plurality of implants is ball-shaped.

23. A kit according to claim 10, wherein the median portion of each of the plurality of implants is ball-shaped.

24. A kit according to claim 23, wherein the median portions have transverse dimensions which increase from a given implant of the plurality of implants to a larger size implant of the plurality of implants.

25. A kit for foot arthroplasty comprising a plurality of implants of increasing sizes and a single bit, each implant comprising, starting from a median portion, a proximal stem and a distal stem shorter than the proximal stem,
these stems and this bit having equal conical tapers between 2° and 6°,
wherein the distal stem of an implant has the same length as the proximal stem of another implant.

26. A kit according to claim 25, wherein said same value is 4.8°.

27. A kit according to claim 25, wherein the bit comprises a working stem provided with at least one visual marker defining, from a point of the bit, a penetration distance equal to the length of at least one distal or proximal stem of an implant of the plurality of implants.

28. A kit according to claim 25, wherein each of the plurality of implants, except for the largest, when classified in increasing order of size, have the proximal stem which has a length equal to that of the distal stem of the next implant.

29. A kit according to claim 25, wherein the median portion of each of the plurality of implants is ball-shaped.
